(19) United States
(12) Patent Application Publication
(10) Pub. No.: US 2025/0373081 A1
(43) Pub. Date: Dec. 4, 2025

(54) CHARGING DEVICE FOR IMPLANT

(71) Applicant: Cochlear Limited, Macquarie University, NSW (AU)

(72) Inventors: Werner Meskens, Opwijk (BE); Carl Van Himbeeck, Mechelen (BE); Padraig Hurley, Frenches Forest (AU); Nathan Isaacson, Balgowlah (AU)

(21) Appl. No.: 18/707,080

(22) PCT Filed: Dec. 1, 2022
(86) PCT No.: PCT/IB2022/061621

§ 371 Date: May 2, 2024

Related U.S. Application Data
(60) Provisional application No. 63/287,313, filed on Dec. 8, 2021.

(51) Int. Cl. H02J 50/12; A61N 1/36; A61N 1/372; A61N 1/378; H02J 50/20; H02J 50/90

(52) U.S. Cl. CPC H02J 50/12; A61N 1/37229; A61N 1/3787; H02J 50/20; H02J 50/90; A61N 1/36038

(57) ABSTRACT

An apparatus includes a housing external to a recipient's body, at least one energy transmission coil on or within the housing, and at least one magnetic material on or within the housing. The housing is configured to be placed on the recipient's body with the at least one energy transmission coil inductively coupled to at least one energy reception coil of a device implanted within the recipient's body and the at least one magnetic material closer to the implanted device than is the at least one energy transmission coil.

### BACKGROUND

### Field

The present application relates generally to systems and methods for charging a device implanted on or within a recipient's body.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises a housing external to a recipient's body, at least one energy transmission coil on or within the housing, and at least one magnetic material on or within the housing. The housing is configured to be placed on the recipient's body with the at least one energy transmission coil inductively coupled to at least one energy reception coil of a device implanted within the recipient's body and the at least one magnetic material closer to the implanted device than is the at least one energy transmission coil.

In another aspect disclosed herein, an apparatus comprises a first housing comprising a portion configured to be placed in contact with a recipient's skin. The apparatus further comprises at least one magnetic material configured to attract to an implanted device beneath the skin by a force configured to hold the portion in contact with the skin. The apparatus further comprises at least one energy transmission coil on or within the first housing and configured to transfer energy to at least one energy reception coil of the implanted device. Upon the portion being held in contact with the skin by the at least one magnetic material, the at least one energy transmission coil is spaced a first distance from the implanted device and the at least one magnetic material is spaced a second distance from the implanted device, the second distance smaller than the first distance.

In another aspect disclosed herein, a method comprises placing a housing comprising a magnetic material and a power transmitting coil over a portion of a recipient's tissue overlying a power receiving coil beneath the tissue. The housing is placed such that a region between the power transmitting coil and the recipient's tissue comprises at least one thermally insulative material, and the magnetic material is closer to the tissue than is the power transmitting coil. The method further comprises wirelessly transferring power from the power transmitting coil to the power receiving coil via a radio-frequency (RF) link.

### BRIEF DESCRIPTION OF THE DRAWINGS

Implementations are described herein in conjunction with the accompanying drawings, in which:

FIG. 1A is a perspective view of an example cochlear implant auditory prosthesis implanted in a recipient in accordance with certain implementations described herein;

FIG. 1B is a perspective view of an example fully implantable middle ear implant auditory prosthesis implanted in a recipient in accordance with certain implementations described herein;

FIG. 1C schematically illustrates a side cross-sectional view of an example transcutaneous system comprising an implantable component and an external component;

FIGS. 2A-2C schematically illustrate cross-sectional views of three example transcutaneous systems each comprising an apparatus compatible with certain implementations described herein;

FIG. 3A is a plot of a cross-sectional view of an example simulation of magnetic field lines in a plane substantially perpendicular to a bottom lower surface of an external device in contact with an outer surface of the recipient's skin;

FIGS. 3B-3D are plots of cross-sectional views of example simulations of magnetic field lines in a plane substantially perpendicular to the first outer surface of the apparatus in contact with an outer surface of the recipient's skin for three example apparatus in accordance with certain implementations described herein;

FIG. 4 shows to plots of a simulation of the magnetic coupling factor for circular coils in accordance with certain implementations described herein as a function of distance from a typical internal communication coil;

FIG. 5 schematically illustrates an example system configured to be worn on a recipient's body and comprising at least one energy transmission coil and at least one magnetic material in accordance with certain implementations described herein; and

FIG. 6 is a flow diagram of an example method in accordance with certain implementations described herein.

### DETAILED DESCRIPTION

Certain implementations described herein provide an external charger configured to be placed on the recipient's skin and to provide fast energy transfer (e.g., charging) to an implant battery while (i) operating within to predetermined safety limitations of thermal heating of the recipient's skin and specific absorption rate (SAR) of human exposure to radio-frequency radiation, (ii) providing sufficient magnetic attraction to the underlying implant, (iii) without excessive heating of the driver circuitry due to switching current or conductive losses, and/or (iv) without major efficiency reduction due to low Q factors. The external charger is configured to maintain a thermally insulating separation (e.g., air gap) between the energy-transmitting coil of the charger and the recipient's skin. Besides providing thermal insulation between the energy-transmitting coil and the recipient's skin, the gap separates the energy-transmitting coil from the recipient such that the energy-transmitting coil is close to loosely inductively coupled to an energy-receiving coil of the implant (e.g., having a magnetic coupling factor between the energy-transmitting coil and the energy-receiving coil to be below 0.2 or to be equal to or less than 0.1) and reducing the SAR of the fast energy transfer. SAR is created by the induced eddy currents on conductive tissue and implant materials from the magnetic flux generated by the energy transmission coil (e.g., Lenz law). Eddy currents that flow inside conductive materials generate heat.

The teachings detailed herein are applicable, in at least some implementations, to any type of implantable or non-implantable stimulation system or device (e.g., implantable or non-implantable auditory prosthesis device or system). Implementations can include any type of medical device that can utilize the teachings detailed herein and/or variations thereof. Furthermore, while certain implementations are described herein in the context of auditory prosthesis devices, certain other implementations are compatible in the context of other types of devices or systems (e.g., smart phones; smart speakers).

Merely for ease of description, apparatus and methods disclosed herein are primarily described with reference to an illustrative medical device, namely an implantable transducer assembly including but not limited to: electro-acoustic electrical/acoustic systems, cochlear implant devices, implantable hearing aid devices, middle ear implant devices, bone conduction devices (e.g., active bone conduction devices; passive bone conduction devices, percutaneous bone conduction devices; transcutaneous bone conduction devices), Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Implementations can include any type of auditory prosthesis that can utilize the teachings detailed herein and/or variations thereof. Certain such implementations can be referred to as “partially implantable,” “semi-implantable,” “mostly implantable,” “fully implantable,” or “totally implantable” auditory prostheses. In some implementations, the teachings detailed herein and/or variations thereof can be utilized in other types of prostheses beyond auditory prostheses.

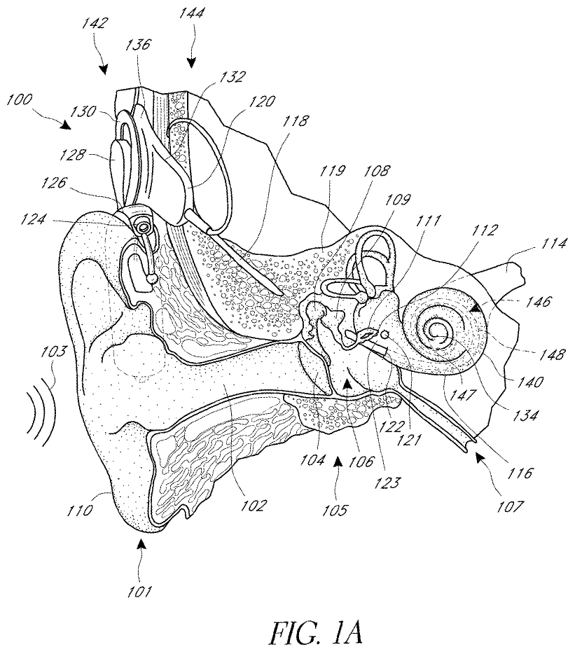

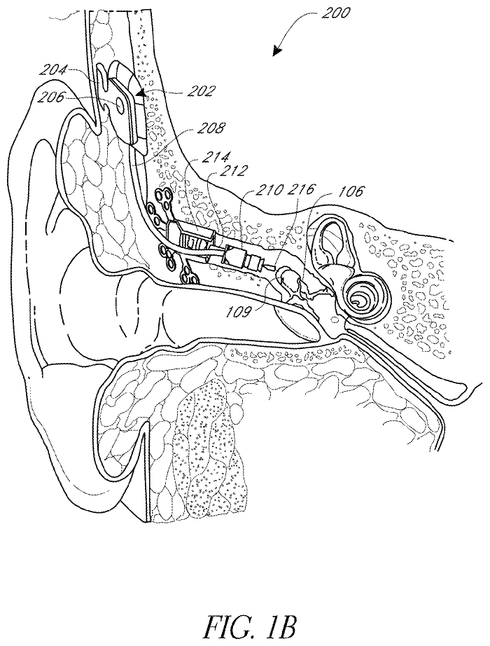

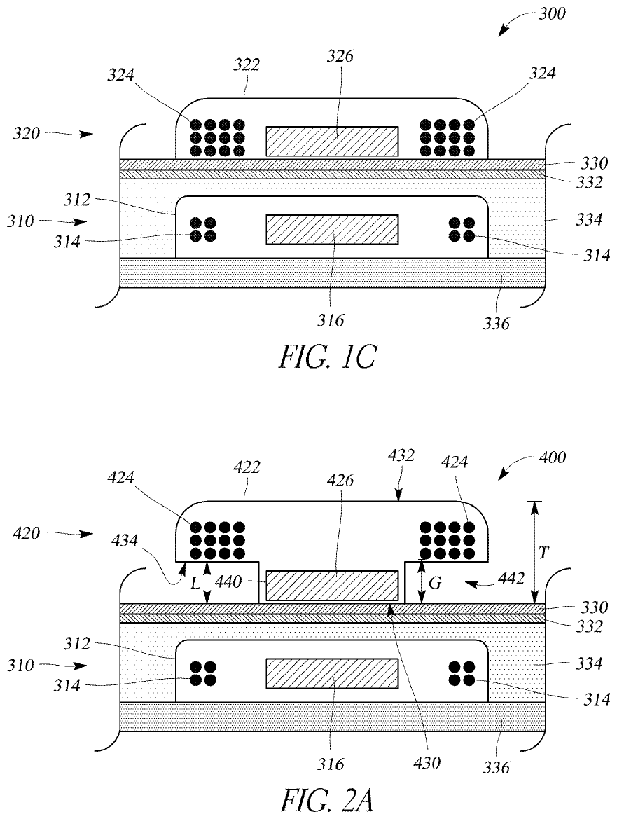

FIG. 1A is a perspective view of an example cochlear implant auditory prosthesis 100 implanted in a recipient in accordance with certain implementations described herein. The example auditory prosthesis 100 is shown in FIG. 1A as comprising an implanted stimulator unit 120 and a microphone assembly 124 that is external to the recipient (e.g., a partially implantable cochlear implant). An example auditory prosthesis 100 (e.g., a totally implantable cochlear implant; a mostly implantable cochlear implant) in accordance with certain implementations described herein can replace the external microphone assembly 124 shown in FIG. 1A with a subcutaneously implantable microphone assembly, as described more fully herein. In certain implementations, the example cochlear implant auditory prosthesis 100 of FIG. 1A can be in conjunction with a reservoir of liquid medicament as described herein.

As shown in FIG. 1A, the recipient has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1A, the example auditory prosthesis 100 comprises one or more components which are temporarily or permanently implanted in the recipient. The example auditory prosthesis 100 is shown in FIG. 1A with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more sound input elements (e.g., an external microphone 124) for detecting sound, a sound processing unit 126 (e.g., disposed in a Behind-The-Ear unit), a power source (not shown), and an external transmitter unit 128. In the illustrative implementations of FIG. 1A, the external transmitter unit 128 comprises an external coil 130 (e.g., a wire antenna coil comprising multiple turns of electrically insulated single-strand or multi-strand platinum or gold wire) and, preferably, a magnet (not shown) secured directly or indirectly to the external coil 130. The external coil 130 of the external transmitter unit 128 is part of an inductive radio frequency (RF) communication link with the internal component 144. The sound processing unit 126 processes the output of the microphone 124 that is positioned externally to the recipient's body, in the depicted implementation, by the recipient's auricle 110. The sound processing unit 126 processes the output of the microphone 124 and generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate electrode assembly 118. In some implementations, the internal receiver unit 132 and the stimulator unit 120 are hermetically sealed within a biocompatible housing. The internal receiver unit 132 comprises an internal coil 136 (e.g., a wire antenna coil comprising multiple turns of electrically insulated single-strand or multi-strand platinum or gold wire), and preferably, a magnet (also not shown) fixed relative to the internal coil 136. The internal receiver unit 132 and the stimulator unit 120 are hermetically sealed within a biocompatible housing, sometimes collectively referred to as a stimulator/receiver unit. The internal coil 136 receives power and/or data signals from the external coil 130 via a transcutaneous energy transfer link (e.g., an inductive RF link). The stimulator unit 120 generates electrical stimulation signals based on the data signals, and the stimulation signals are delivered to the recipient via the elongate electrode assembly 118.

The elongate electrode assembly 118 has a proximal end connected to the stimulator unit 120, and a distal end implanted in the cochlea 140. The electrode assembly 118 extends from the stimulator unit 120 to the cochlea 140 through the mastoid bone 119. In some implementations, the electrode assembly 118 may be implanted at least in the basal region 116, and sometimes further. For example, the electrode assembly 118 may extend towards apical end of cochlea 140, referred to as cochlea apex 134. In certain circumstances, the electrode assembly 118 may be inserted into the cochlea 140 via a cochleostomy 122. In other circumstances, a cochleostomy may be formed through the round window 121, the oval window 112, the promontory 123, or through an apical turn 147 of the cochlea 140.

The elongate electrode assembly 118 comprises a longitudinally aligned and distally extending array 146 of electrodes or contacts 148, sometimes referred to as electrode or contact array 146 herein, disposed along a length thereof. Although the electrode array 146 can be disposed on the electrode assembly 118, in most practical applications, the electrode array 146 is integrated into the electrode assembly 118 (e.g., the electrode array 146 is disposed in the electrode assembly 118). As noted, the stimulator unit 120 generates stimulation signals which are applied by the electrodes 148 to the cochlea 140, thereby stimulating the auditory nerve 114.

While FIG. 1A schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

FIG. 1B schematically illustrates a perspective view of an example fully implantable auditory prosthesis 200 (e.g., fully implantable middle ear implant or totally implantable acoustic system), implanted in a recipient, utilizing an acoustic actuator in accordance with certain implementations described herein. The example auditory prosthesis 200 of FIG. 1B comprises a biocompatible implantable assembly 202 (e.g., comprising an implantable capsule) located subcutaneously (e.g., beneath the recipient's skin and on a recipient's skull). While FIG. 1B schematically illustrates an example implantable assembly 202 comprising a microphone, in other example auditory prostheses 200, a pendant microphone can be used (e.g., connected to the implantable assembly 202 by a cable). The implantable assembly 202 includes a signal receiver 204 (e.g., comprising a coil element) and an acoustic transducer 206 (e.g., a microphone comprising a diaphragm and an electret or piezoelectric transducer) that is positioned to receive acoustic signals through the recipient's overlying tissue. The implantable assembly 202 may further be utilized to house a number of components of the fully implantable auditory prosthesis 200. For example, the implantable assembly 202 can include an energy storage device and a signal processor (e.g., a sound processing unit). Various additional processing logic and/or circuitry components can also be included in the implantable assembly 202 as a matter of design choice.

For the example auditory prosthesis 200 shown in FIG. 1B, the signal processor of the implantable assembly 202 is in operative communication (e.g., electrically interconnected via a wire 208) with an actuator 210 (e.g., comprising a transducer configured to generate mechanical vibrations in response to electrical signals from the signal processor). In certain implementations, the example auditory prosthesis 100, 200 shown in FIGS. 1A and 1B can comprise an implantable microphone assembly, such as the microphone assembly 206 shown in FIG. 1B. For such an example auditory prosthesis 100, the signal processor of the implantable assembly 202 can be in operative communication (e.g., electrically interconnected via a wire) with the microphone assembly 206 and the stimulator unit of the main implantable component 120. In certain implementations, at least one of the microphone assembly 206 and the signal processor (e.g., a sound processing unit) is implanted on or within the recipient.

The actuator 210 of the example auditory prosthesis 200 shown in FIG. 1B is supportably connected to a positioning system 212, which in turn, is connected to a bone anchor 214 mounted within the recipient's mastoid process (e.g., via a hole drilled through the skull). The actuator 210 includes a connection apparatus 216 for connecting the actuator 210 to the ossicles 106 of the recipient. In a connected state, the connection apparatus 216 provides a communication path for acoustic stimulation of the ossicles 106 (e.g., through transmission of vibrations from the actuator 210 to the incus 109).

During normal operation, ambient acoustic signals (e.g., ambient sound) impinge on the recipient's tissue and are received transcutaneously at the microphone assembly 206. Upon receipt of the transcutaneous signals, a signal processor within the implantable assembly 202 processes the signals to provide a processed audio drive signal via wire 208 to the actuator 210. As will be appreciated, the signal processor may utilize digital processing techniques to provide frequency shaping, amplification, compression, and other signal conditioning, including conditioning based on recipient-specific fitting parameters. The audio drive signal causes the actuator 210 to transmit vibrations at acoustic frequencies to the connection apparatus 216 to affect the desired sound sensation via mechanical stimulation of the incus 109 of the recipient.

The subcutaneously implantable microphone assembly 202 is configured to respond to auditory signals (e.g., sound; pressure variations in an audible frequency range) by generating output signals (e.g., electrical signals; optical signals; electromagnetic signals) indicative of the auditory signals received by the microphone assembly 202, and these output signals are used by the auditory prosthesis 100, 200 to generate stimulation signals which are provided to the recipient's auditory system. To compensate for the decreased acoustic signal strength reaching the microphone assembly 202 by virtue of being implanted, the diaphragm of an implantable microphone assembly 202 can be configured to provide higher sensitivity than are external non-implantable microphone assemblies. For example, the diaphragm of an implantable microphone assembly 202 can be configured to be more robust and/or larger than diaphragms for external non-implantable microphone assemblies.

The example auditory prostheses 100 shown in FIG. 1A utilizes an external microphone 124 and the auditory prosthesis 200 shown in FIG. 1B utilizes an implantable microphone assembly 206 comprising a subcutaneously implantable acoustic transducer. In certain implementations described herein, the auditory prosthesis 100 utilizes one or more implanted microphone assemblies on or within the recipient. In certain implementations described herein, the auditory prosthesis 200 utilizes one or more microphone assemblies that are positioned external to the recipient and/or that are implanted on or within the recipient, and utilizes one or more acoustic transducers (e.g., actuator 210) that are implanted on or within the recipient. In certain implementations, an external microphone assembly can be used to supplement an implantable microphone assembly of the auditory prosthesis 100, 200. Thus, the teachings detailed herein and/or variations thereof can be utilized with any type of external or implantable microphone arrangement, and the acoustic transducers shown in FIGS. 1A and 1B are merely illustrative.

FIG. 1C schematically illustrates a side cross-sectional view of an example transcutaneous system 300 comprising an implantable component 310 and an external component 320. For example, the transcutaneous system 300 can comprise an auditory prosthesis system in which the implantable component 310 comprises one or more active elements (e.g., stimulator unit 120; assembly 202; vibrating actuator; not shown in FIG. 1C) configured to deliver stimuli to the recipient's body.

The implantable component 310 comprises at least one implantable housing 312 configured to be positioned beneath tissue of the recipient's body. For example, as shown in FIG. 1C, the at least one implantable housing 312 is beneath the skin 330, fat 332, and/or muscular 334 layers and above a bone 336 (e.g., skull) in a portion of the recipient's body (e.g., the head). The at least one implantable housing 312 contains at least one internal energy reception coil 314 (e.g., a planar electrically conductive wire with multiple windings) and at least one internal magnetic (e.g., ferromagnetic; ferrimagnetic; permanent magnet) material 316 (e.g., disk; plate) positioned within a region at least partially bounded by the at least one internal energy reception coil 314. The at least one internal magnetic material 316 can comprise a diamagnetic magnet configured to be compatible with magnetic resonance imaging of the recipient. The at least one internal magnetic material 316 is configured to establish a magnetic attraction between the external component 320 and the implantable component 310 sufficient to hold the external component 320 against an outer surface of the skin 330. The at least one implantable housing 312 can comprise a first portion configured to contain the at least one internal energy reception coil 314 and the at least one internal magnetic material 316 and a second portion configured to contain the one or more active elements, or the at least one implantable housing 312 can comprise a single housing portion configured to contain the at least one internal energy reception coil 314, the at least one internal magnetic material 316, and the one or more active elements.

The external component 320 comprises an external housing 322 configured to be positioned on an outer surface of the skin 330 and contains at least one external energy transmission coil 324 (e.g., a planar electrically conductive wire with multiple windings) and at least one external magnetic (e.g., ferromagnetic; ferrimagnetic; permanent magnet) material 326 (e.g., disk; plate) positioned within a region at least partially bounded by the at least one external energy transmission coil 324. The at least one external magnetic material 326 is configured to establish a magnetic attraction between the external component 320 and the implantable component 310 sufficient to hold the external component 320 against the outer surface of the skin 330. To produce a sufficiently strong magnetic attraction, the at least one external magnetic material 326 is positioned as close as possible to the outer surface of the recipient's skin 330 (e.g., as close as possible to a surface of the external housing 322 that contacts the recipient's skin 330), thereby minimizing the distance between the at least one external magnetic material 326 and the at least one internal magnetic material 316.

The at least one external energy transmission coil 324 is configured to be in wireless electrical communication (e.g., via a radio-frequency or RF link) with the at least one internal energy reception coil 314 when the external component 320 is positioned on the skin 330 of the recipient above the internal component 310 (e.g., the external component 320 being held in place by the magnetic attraction between the at least one internal magnetic material 316 and the at least one external magnetic material 326). For example, the at least one external energy transmission coil 324 can be inductively coupled with the at least one internal energy reception coil 314 and configured to wirelessly transmit electrical power to the at least one internal energy reception coil 314 and/or configured to wirelessly transmit information (e.g., data signals; control signals) to and/or to wirelessly receive information from the at least one internal energy reception coil 314.

Conventionally, the at least one external energy transmission coil 324 is configured to be as close as possible to the at least one internal energy reception coil 314 to maximize the strength of the inductive coupling of the at least one external energy transmission coil 324 with the at least one internal energy reception coil 314. For example, as shown in FIG. 1C, both the at least one external magnetic material 326 and the at least one external energy transmission coil 324 are positioned the same distance from the implantable component 310 (e.g., with little or no spacing between the at least one external energy transmission coil 324 and the surface of the external housing 322 that contacts the recipient's skin 330). Such configurations work well for systems that use the at least one external energy transmission coil 324 for wirelessly transmitting information to and/or wirelessly receiving information from the at least one internal energy reception coil 314. However, for systems that use the at least one external energy transmission coil 324 for wirelessly transferring electrical power to the at least one internal energy reception coil 314, such configurations can result in heat above a predetermined thermal threshold and/or electromagnetic radiation or magnetic emissions above a predetermined specific absorption rate (SAR) threshold, the thermal and/or SAR thresholds corresponding to discomfort, pain, and/or damage to the recipient.

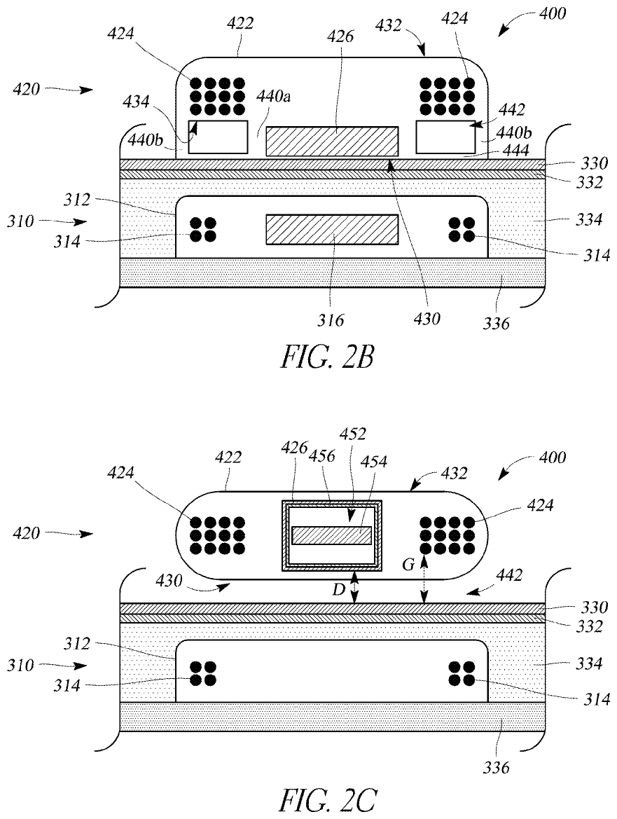

FIGS. 2A-2C schematically illustrate cross-sectional views of three example transcutaneous systems 400 each comprising an implantable component 310 and an apparatus 420 (e.g., external component of the transcutaneous system 400) compatible with certain implementations described herein. The apparatus 420 of certain implementations is configured to be positioned outside a recipient's body and in wireless communication with an implantable component 310 implanted within the recipient's body. In certain implementations, the apparatus 420 comprises a housing 422 external to the recipient's body, at least one energy transmission coil 424 on or within the housing 422, and at least one magnetic material 426 on or within the housing 422. The housing 422 is configured to be placed on the recipient's body (e.g., on or over the recipient's skin 330) with the at least one energy transmission coil 424 inductively coupled to at least one energy reception coil of a device (e.g., at least one internal energy reception coil 314 of an implantable component 310) implanted within the recipient's body and the at least one magnetic material 426 closer to the implanted device than is the at least one energy transmission coil 424.

In certain implementations, the at least one energy transmission coil 424 comprises multiple turns of electrically insulated single-strand or multi-strand copper wire (e.g., a planar electrically conductive wire with multiple windings having a substantially circular, rectangular, spiral, or oval shape or other shape) or copper traces on epoxy of a printed circuit board. The at least one energy transmission coil 424 can have a diameter, length, and/or width (e.g., along a lateral direction substantially parallel to the recipient's skin 330) less than or equal to 40 millimeters (e.g., in a range of 15 millimeters to 35 millimeters; in a range of 25 millimeters to 35 millimeters; in a range of less than 30 millimeters; in a range of 15 millimeters to 30 millimeters). When the apparatus 420 is positioned on or over the skin 330 of the recipient above the internal component 310 (e.g., the apparatus 420 of FIGS. 2A-2B being held in place by the magnetic attraction between the at least one internal magnetic material 316 and the at least one magnet 426 or the apparatus 420 of FIG. 2C is held in place behind the recipient's ear by a hook portion of the housing 422), the at least one energy transmission coil 424 is configured to wirelessly transmit electrical power to the at least one internal energy reception coil 314 (e.g., via a radio-frequency or RF link).

In certain implementations, the at least one magnetic material 426 comprises a ferromagnetic material, a ferrimagnetic material, and/or a permanent magnet (e.g., disk; plate) positioned within the housing 422. For example, as schematically illustrated by FIGS. 2A and 2B, the at least one magnetic material 426 can be configured to establish a magnetic attraction between the apparatus 420 and the implantable component 310 (e.g., generate a magnetic attractive force with the at least one internal magnetic material 316 of the implanted device 310) sufficient to hold the apparatus 420 (e.g., housing 422) on the recipient's body (e.g., against the outer surface of the skin 330). To produce a sufficiently strong magnetic attraction, the at least one magnetic material 426 is positioned as close as possible to the outer surface of the recipient's skin 330 (e.g., as close as possible to a first outer surface 430 of the housing 422 that contacts the recipient's skin 330), thereby minimizing the distance between the at least one magnetic material 426 and the at least one internal magnetic material 316. For another example, as schematically illustrated by FIG. 2C, the at least one magnetic material 426 (e.g., a ferrite sheet) substantially bounds a region 452 containing circuitry 454 (e.g., a printed-circuit board with electrically conductive traces and power and ground planes, and electrical components), the at least one magnetic material 426 can be configured to redirect magnetic flux generated by the at least one energy transmission coil 424 from entering the region 452. As shown in FIG. 2C, the region 452 can also include an electrically conductive electromagnetic interference shield 456 substantially surrounding the circuitry 454.

In certain implementations, the housing 422 is configured to hermetically seal the at least one energy transmission coil 424 and/or the at least one magnetic material 426 from an environment surrounding the housing 422. The housing 422 of certain implementations comprises at least one biocompatible material (e.g., skin-friendly) that is substantially transparent to the electromagnetic or magnetic fields generated by the at least one energy transmission coil 424 such that the housing 422 does not substantially interfere with the transmission of power via magnetic induction between the apparatus 420 and the implanted device.

The housing 422 can have a width (e.g., along a lateral direction substantially parallel to the recipient's skin 330) less than or equal to 40 millimeters (e.g., in a range of 15 millimeters to 35 millimeters; in a range of 25 millimeters to 35 millimeters; in a range of less than 30 millimeters; in a range of 15 millimeters to 30 millimeters). The housing 422 can have a thickness T (e.g., between the first outer surface 430 configured to contact the recipient's skin 330 and a second outer surface 432 on an opposite side of the housing 422 from the first outer surface 430), the thickness T less than or equal to 10 millimeters (e.g., in a range of less than or equal to 7 millimeters, in a range of less than or equal to 6 millimeters; in a range of less than or equal to 5 millimeters).

In certain implementations, as schematically illustrated by FIGS. 2A-2B, the housing 422 comprises at least one protrusion 440 (e.g., extending from a third outer surface 434 to the first outer surface 430). The at least one protrusion 440 is configured to form a gap 442 between the at least one energy transmission coil 424 and the recipient's body (e.g., skin 330). In certain other implementations, as schematically illustrated by FIG. 2C, the housing 422 does not comprise a protrusion 440 and but is configured to be held over the outer surface of the recipient's skin 330 (e.g., held behind the recipient's ear by a hook portion) with the gap 442 between the at least one energy transmission coil 424 and the recipient's body (e.g., skin 330). The gap 442 is configured to provide thermal insulation between the at least one energy transmission coil 424 and the recipient's skin 330. At least a portion of the gap 442 can comprise air (e.g., at least some of the air configured to flow between the apparatus 420 and the recipient's skin 330) or a thermally isolating material (e.g., aerogel; foam).

For example, the at least one protrusion 440 of FIGS. 2A and 2B can have a length L (e.g., from the first outer surface 430 to the third outer surface 434) of at least 1 millimeter (e.g., at least 2 millimeters; at least 5 millimeters) and/or the gap 442 can have a thickness G (e.g., from the first outer surface 430 to the at least one energy transmission coil 424) of at least 1 millimeter (e.g., at least 2 millimeters; at least 5 millimeters). In certain implementations, the housing 422 is configured to controllably adjust the protrusion length L and/or the gap thickness G (e.g., by controllably extending and/or retracting the first outer surface 430 relative to the third outer surface 434; by using a mechanical slider). For another example, the at least one magnetic material 426 of FIG. 2C can be configured to be held a first distance D of at least 1 millimeter (e.g., at least 2 millimeters; at least 5 millimeters) over the recipient's tissue (e.g., to the outer surface of the recipient's skin 330) and the at least one energy transmission coil 424 can have a second distance (e.g., gap thickness G) of at least 1 millimeter (e.g., at least 2 millimeters; at least 5 millimeters) over the recipient's tissue (e.g., to the outer surface of the recipient's skin 330), the second distance greater than the first distance. In certain implementations in which the recipient's tissue between the at least one energy transmission coil 424 and the at least one internal energy reception coil 314 has a tissue thickness, the distance between the at least one energy transmission coil 424 and the at least one internal energy reception coil 314 is at least 1 millimeter greater than the tissue thickness.

In certain implementations, as schematically illustrated by FIGS. 2A and 2B, the at least one magnetic material 426 is on or within the at least one protrusion 440. For example, the protrusion 440 comprising the at least one magnetic material 426 can be positioned substantially centrally relative to the housing 422 and substantially concentric with the at least one energy transmission coil 424. The protrusion 440 comprising the at least one magnetic material 426 can have a width (e.g., along a lateral direction substantially parallel to the recipient's skin 330) less than or equal to 30 millimeters (e.g., in a range of 10 millimeters to 25 millimeters; in a range of 15 millimeters to 25 millimeters; in a range of less than 20 millimeters; in a range of 10 millimeters to 20 millimeters).

In certain implementations, only the protrusion 440 comprising the at least one magnetic material 426 contacts the recipient's skin 330 (see, e.g., FIG. 2A), such that the first outer surface 430 in contact with the recipient's skin 330 has the same width as does the protrusion 440. In certain other implementations (see, e.g., FIG. 2B), the housing 422 comprises a central protrusion 440a (e.g., central portion of the housing 422) and one or more other thermally insulative protrusions 440b (e.g., peripheral portion of the housing 422 encircling the central portion of the housing 422; thermal insulation) that extend from the third outer surface 434 to a housing portion 444 that comprises the first outer surface 430 in contact with the recipient's skin 330 and that is in mechanical communication with the central protrusion 440a and the one or more other protrusions 440b. The gap 442 comprises channels between the central protrusion 440a and the one or more other protrusions 440b, the channels configured to allow air to flow therethrough. As shown in FIG. 2B, the first outer surface 430 in contact with the recipient's skin 330 has the same width as does the housing 422, which is larger than the width of the central protrusion 440a. By having a larger width, the first outer surface 430 can distribute the magnetic attractive force from the at least one magnetic material 426 across a wider area on the recipient's skin 330, thereby reducing the pressure experienced by the recipient. The one or more other protrusions 440b can be configured to provide structural stability of the housing 422 on the skin 330 (e.g., positioned along a periphery of the housing 422) while not providing a substantially thermally conductive pathway for heat from the at least one energy transmission coil 424 to reach the recipient's skin 330.

In certain implementations (e.g., as shown in FIG. 2C), the at least one magnetic material 426 is configured to shield the region 452 within the at least one magnetic material 426 from the magnetic flux generated by the at least one energy transmission coil 424. In addition, the at least one magnetic material 426 can shield the at least one energy transmission coil 424 from decreases of the coil inductance and/or Q factor that would otherwise (e.g., without the at least one magnetic material 426) be caused by the circuitry 454 encircled by the at least one energy transmission coil 424.

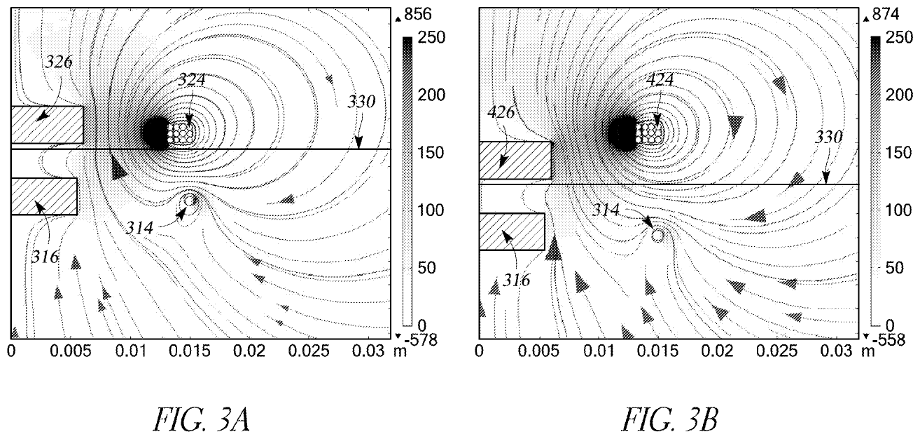

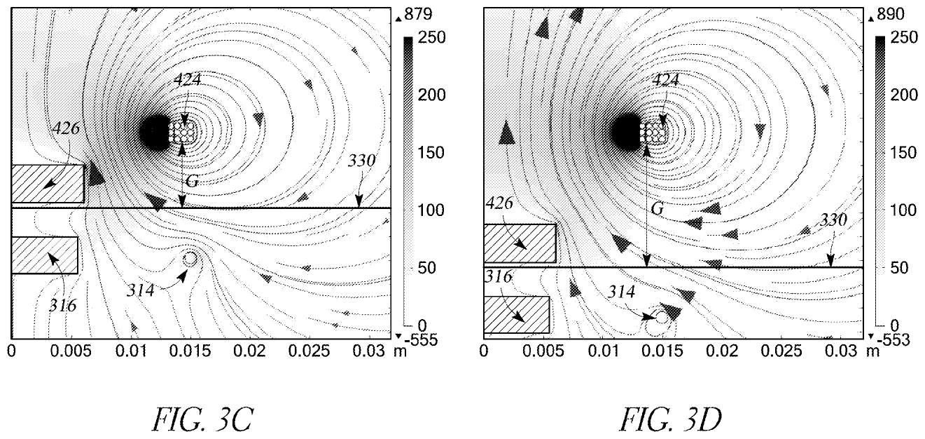

FIG. 3A is a plot of a cross-sectional view of an example simulation of magnetic field lines in a plane substantially perpendicular to a bottom lower surface of an external device 320 in contact with an outer surface of the recipient's skin 330 (see, e.g., FIG. 1C). The left vertical axis of FIG. 3A corresponds to a symmetry axis of the external device 320 extending through the centers of the at least one external magnetic material 326 and the at least one external energy transmission coil 324 of the external component 320, and the at least one internal magnetic material 316 and the at least one internal energy reception coil 314 of the implantable component 310. In FIG. 3A, the at least one external magnetic material 326 is at the same distance from the recipient's skin 330 as is the at least one external energy transmission coil 324 (e.g., spaced from the recipient's skin 330 by a wall thickness of the housing 422). The simulation of FIG. 3A shows that the region having the highest magnetic flux (shown as a black area) is at an inner boundary of the at least one external energy transmission coil 324 and overlaps with the recipient's skin 330. As a result, for the configuration of FIG. 3A, to ensure that the recipient is not exposed to a SAR higher than a predetermined safety threshold, the at least one external energy transmission coil 324 is operated at a sufficiently low power (e.g., slower energy transfer).

FIGS. 3B-3D are plots of cross-sectional views of example simulations of magnetic field lines in a plane substantially perpendicular to the first outer surface 430 of the apparatus 400 in contact with an outer surface of the recipient's skin 330 (see, e.g., FIGS. 2A and 2B) for three example apparatus 400 in accordance with certain implementations described herein. The left vertical axes of the plots of FIGS. 3B-3D correspond to a symmetry axis of the external apparatus 420 extending through the centers of the at least one magnetic material 426 and the at least one energy transmission coil 424 of the apparatus 420, and the at least one internal magnetic material 316 and the at least one internal energy reception coil 314 of the implantable component 310. For each of FIGS. 3B-3D, the implantable device 310 is the same as in FIG. 3A (e.g., the at least one internal energy reception coil 314 and the at least one internal magnetic material 316 are the same as in FIG. 3A), and the at least one energy transmission coil 424 has the same dimensions, materials, and is operated with the same power as the at least one external energy transmission coil 324 of FIG. 3A. In addition, for each of FIGS. 3B-3D, the at least one magnetic material 426 is spaced from the recipient's skin 330 by a wall thickness of the housing 422.

While in FIG. 3A, both the at least one external magnetic material 326 and the at least one external energy transmission coil 324 are equidistant from the recipient's skin 330 (e.g., less than 1 millimeter above the recipient's skin 330), in each of FIGS. 3B-3D, the at least one magnetic material 426 is closer to the implanted device 310 than is the at least one energy transmission coil 424 and is closer to the recipient's tissue (e.g., skin 330) than is the at least one energy transmission coil 424. For example, as shown in FIGS. 3B-3D, while the at least one magnetic material 426 is less than 1 millimeter above the recipient's skin 330, the at least one energy transmission coil 424 is spaced from the recipient's skin 330 by a gap thickness G (e.g., of at least 1 millimeter; at least 2 millimeters; at least 5 millimeters). As the gap thickness G is increased, as shown in FIGS. 3B-3D, the region having the highest magnetic flux (shown as a black area) is at an inner boundary of the at least one energy transmission coil 424 but does not substantially overlap with the recipient's skin 330. In addition, the magnetic flux at the recipient's skin 330 decreases with increasing gap thickness G. As a result, for the configuration of FIGS. 3B-3D, the at least one energy transmission coil 424 can be operated at higher powers (e.g., faster energy transfer) while ensuring that the recipient is not exposed to a SAR higher than a predetermined safety threshold and/or excessive skin and tissue temperature increases.

In certain implementations, the at least one energy transmission coil 424 is configured (e.g., optimized) for substantially faster energy transfer than is provided by conventional communication coils which are configured to be used for both energy transfer and data communications between the external component 320 and the implantable component 310 of a conventional transcutaneous system 300. For example, conventional communication coils that are configured for data transfer over a closely coupled RF link with an internal communication coil typically have magnetic coupling coefficients (k) of 0.2 for sufficiently strong coupling, Q-factors less than 30 to reduce (e.g., avoid) ringing or other effects that can be deleterious to data transfer, and staggered tuning to provide sufficient data integrity. In certain implementations in which the at least one energy transmission coil 424 is not used for data transfer, the at least one energy transmission coil 424 has a magnetic coupling factor (k) less than 0.3 to the at least one internal energy reception coil 314 and/or has a quality (Q) factor in a range of 30 to 250. In certain implementations, the reduction of the magnetic coupling factor (k) is due to the additional spacing between the at least one energy transmission coil 424 and the at least one internal energy reception coil 314 resulting from the gap 442, but both the at least one energy transmission coil 424 and the at least one internal energy reception coil 314 can be tuned to resonant frequencies that are close (e.g., within ±10%) to the operational frequency of the RF energy transfer (e.g., 6.78 MHz).

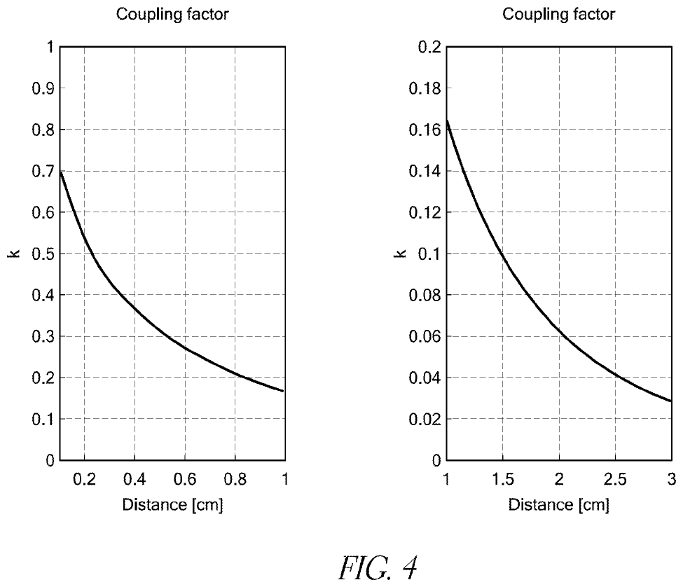

For example, the at least one energy transmission coil 424 can comprise a substantially circular Cu coil of six windings of 0.8 mm-thick wire, the coil having a diameter of 30 millimeters. For operational frequencies in a range of about 4.7-6.8 MHz, such a coil can have an inductance of about 2 μH, an equivalent series resistance of about 400 mΩ, a total impedance of about 70Ω, and a Q-factor of about 200. In certain implementations, the Q-factor is substantially unaffected by the at least one magnetic material 440, which is spaced from the at least one energy transmission coil 424 (e.g., by a z-axis offset as shown in FIGS. 2A and 2B). FIG. 4 shows to plots of a simulation of the magnetic coupling factor (k) for such a circular Cu coil in accordance with certain implementations described herein as a function of distance from a typical internal energy reception coil 314. As seen in FIG. 4, for coil-to-coil distances between the at least one energy transmission coil 424 and the at least one internal energy reception coil 314 greater than about 5 millimeters, the magnetic coupling factor (k) is less than 0.3.

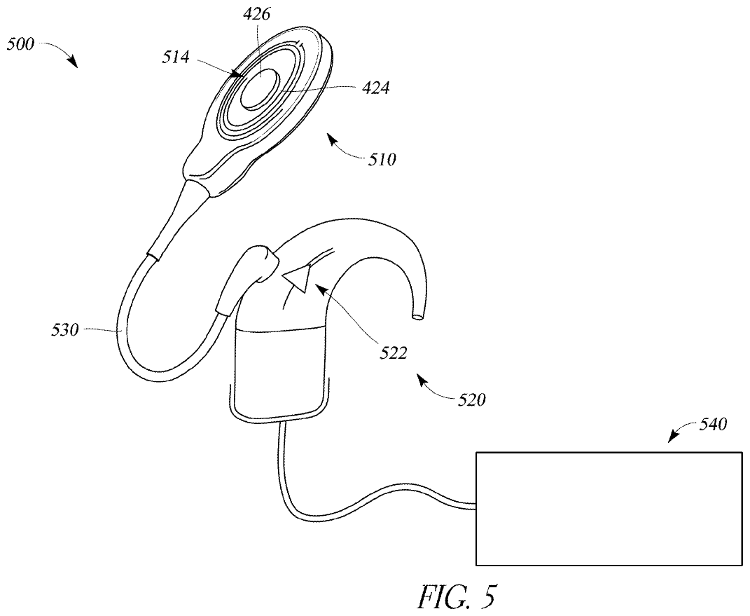

FIG. 5 schematically illustrates an example system 500 configured to be worn on a recipient's body and comprising at least one energy transmission coil 424 and at least one magnetic material 426 in accordance with certain implementations described herein. The system 500 can be configured for fast charging of an implant (e.g., for fast charging a battery of a totally implantable cochlear implant). The system 500 of FIG. 5 comprises a first portion 510 (e.g., first housing; apparatus 420 of FIGS. 2A-2B) comprising the at least one energy transmission coil 424 and the at least one magnetic material 426 and configured to be worn over at least one internal energy receiving coil of the implant, and a second portion 520 (e.g., second housing) separate from the first portion 510 and configured to be worn over a recipient's ear (e.g., a “behind-the-ear” or “on-the-go” sound processor). The at least one energy transmission coil 424 is operationally coupled (e.g., electrically connected; by a differential pair of electrical conductors 530) to driver circuitry 522 within the second portion 520.

In certain implementations, the driver circuitry 522 comprises low loss driver circuitry (e.g., class-E radio-frequency power amplifier; GaN MOSFET). The efficiency of the power transfer at lower magnetic coupling factors (k) can be compensated by higher Q factors of the coils and/or the low loss driver circuitry. The higher Q factors can also reduce harmonics emanating from the apparatus 420, thereby improving the electromagnetic compatibility (EMC) of the system 400. In certain implementations, the at least one energy transmission coil 424 and the at least one internal energy receiving coil of the implant are substantially optimized such that the apparatus 420 can be operated without excessive heating of the driver circuitry 522 due to switching current or conductive losses. In certain implementations, the system 500 can be operated at the carrier frequency such that staggered tuning is not used (e.g., when reflected impedance causes only little or no frequency shift). Certain such implementations are compatible for use by an apparatus 420 comprising a charger upon which the recipient can lay a portion of the recipient's body comprising the implantable component 410 (e.g., a pillow charger upon which the recipient can lay the recipient's head to charge an implanted portion of an auditory prosthesis).

The second portion 520 of certain implementations can further comprise one or more microprocessors (e.g., application-specific integrated circuits; generalized integrated circuits programmed by software with computer executable instructions; microelectronic circuitry; microcontrollers) configured to control operation of the system 500 (e.g., set or adjust parameters of the energy transfer in response to user input and/or conditions during operation). In certain implementations, the one or more microprocessors comprise and/or are in operative communication with at least one storage device (e.g., at least one tangible or non-transitory computer readable storage medium; read only memory; random access memory; flash memory) configured to store information (e.g., data; commands) accessed by the one or more microprocessors during operation. The at least one storage device can be encoded with software (e.g., a computer program downloaded as an application) comprising computer executable instructions for instructing the one or more microprocessors (e.g., executable data access logic, evaluation logic, and/or information outputting logic). In certain implementations, the one or more microprocessors execute the instructions of the software to provide functionality as described herein.

In certain implementations, the system 500 further comprises tuning circuitry 514 (e.g., at least one capacitor and/or inductor) configured to be adjusted to tune a resonant frequency of the at least one energy transmission coil 424. In certain implementations, the tuning circuitry 514 is within the first portion 510 (as shown in FIG. 5), while in certain other implementations, the tuning circuitry 514 is within the driver circuitry 522 of the second portion 520. In certain implementations, the driver circuitry 522 of the second portion 520 is configured to be releasably coupled (e.g., attachable and detachable) with an external battery 540.

In certain implementations, the system 500 further comprises at least one sensor (e.g., accelerometer; gyroscope) configured to generate signals indicative of a position and/or an orientation of the first housing 510 and the driver circuitry 522 is configured to receive the signals and to adjust operation of the at least one energy transmission coil 424 in response to the signals. For example, to ensure that the apparatus 420 schematically illustrated by FIGS. 2A and 2B is positioned with the at least one magnetic material 426 closer to the implantable component 310 than is the at least one energy transmission coil 424, the driver circuitry 522 can be configured to controllably disable operation of the at least one energy transmission coil 424 upon detecting that the apparatus 420 is positioned incorrectly (e.g., upside-down) relative to the implantable component 310. In certain other implementations (e.g., as schematically illustrated by FIG. 2C), the apparatus 420 is configured to be operated with either the first outer surface 430 or the second outer surface 432 closer to the implantable component 310 (e.g., the apparatus 420 hanging behind either of the recipient's ears).

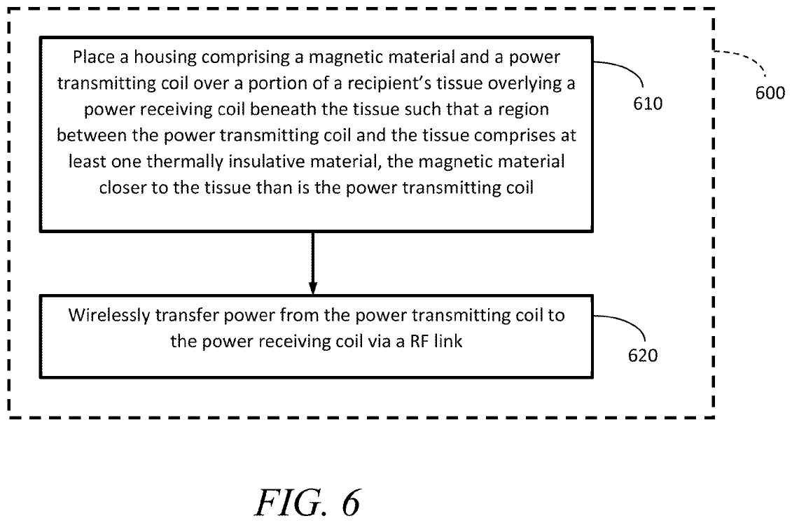

FIG. 6 is a flow diagram of an example method 600 in accordance with certain implementations described herein. While the method 600 is described by referring to some of the structures of the example apparatus 400 of FIGS. 2A-2C, 3B-3D, and 5, other apparatus and systems with other configurations of components can also be used to perform the method 600 in accordance with certain implementations described herein.

In an operational block 610, the method 600 comprises placing a housing 422 comprising a magnetic material (e.g., at least one magnetic material 426) and a power transmitting coil (e.g., at least one energy transmission coil 424) over a portion of a recipient's tissue overlying a power receiving coil (e.g., at least one energy reception coil 314) beneath the tissue such that a region between the power transmitting coil and the recipient's tissue comprises at least one thermally insulative material (e.g., gap 442 comprising air; thermal insulation material), the magnetic material closer to the tissue than is the power transmitting coil. In an operational block 620, the method 600 further comprises wirelessly transferring power from the power transmitting coil to the power receiving coil via a radio-frequency (RF) link. In certain implementations, a distance between the power transmitting coil and the power receiving coil is at least one millimeter greater than a thickness of the tissue between the magnetic material and the power receiving coil. In certain implementations, a first distance between the magnetic material and an outer surface of the portion of the recipient's tissue overlying the power receiving coil is less than a second distance between the power transmitting coil and the outer surface of the portion of the recipient's tissue overlying the power receiving coil.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of various devices, various implementations described herein can be incorporated in a variety of other suitable devices, methods, and contexts. More generally, as can be appreciated, certain implementations described herein can be used in a variety of implantable medical device contexts that can benefit from certain attributes described herein.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein but should be defined only in accordance with the claims and their equivalents.

1. An apparatus comprising:
a housing external to a recipient's body;
at least one energy transmission coil on or within the housing; and
at least one magnetic material on or within the housing, the housing configured to be placed on the recipient's body with the at least one energy transmission coil inductively coupled to at least one energy reception coil of a device implanted within the recipient's body and the at least one magnetic material closer to the implanted device than is the at least one energy transmission coil.

2. The apparatus of claim 1, wherein the housing comprises at least one protrusion configured to form a gap between the at least one energy transmission coil and the recipient's body.

3. The apparatus of claim 2, wherein the at least one magnetic material is on or within a protrusion of the at least one protrusion positioned substantially centrally relative to the housing.

4. The apparatus of claim 2, wherein the gap has a thickness of at least 1 millimeter.

5. The apparatus of claim 4, wherein the housing is configured to controllably adjust the thickness of the gap.

6. The apparatus of claim 2, wherein the gap comprises air.

7. The apparatus of claim 1, wherein the at least one energy transmission coil is substantially planar and at least a portion of the at least one magnetic material is in a region between the at least one energy transmission coil and the at least one energy reception coil.

8. The apparatus of claim 7, wherein tissue of the recipient's body between the at least one energy transmission coil and the at least one energy reception coil has a tissue thickness and a distance between the at least one energy transmission coil and the at least one energy reception coil is at least 1 millimeter greater than the tissue thickness.

9. The apparatus of claim 1, wherein the at least one energy transmission coil has a quality factor in a range of 30 to 250.

10. The apparatus of claim 1, wherein the at least one energy transmission coil has a magnetic coupling factor to the at least one energy reception coil, the magnetic coupling factor less than 0.3.

11. The apparatus of claim 1, wherein the at least one magnetic material is configured to generate a magnetic attractive force with at least a portion of the implanted device, the force sufficient to hold the housing on the recipient's body.

12. The apparatus of claim 1, wherein the at least one magnetic material substantially bounds a region containing circuitry and is configured to redirect magnetic flux generated by the at least one energy transmission coil from entering the region.

13. An apparatus comprising:
a first housing comprising a portion configured to be placed in contact with a recipient's skin;
at least one magnetic material configured to attract to an implanted device beneath the skin by a force configured to hold the portion in contact with the skin; and
at least one energy transmission coil on or within the first housing and configured to transfer energy to at least one energy reception coil of the implanted device, wherein upon the portion being held in contact with the skin by the at least one magnetic material, the at least one energy transmission coil is spaced a first distance from the implanted device and the at least one magnetic material is spaced a second distance from the implanted device, the second distance smaller than the first distance.

14. The apparatus of claim 13, wherein a region between the at least one energy transmission coil and the skin comprises at least one thermally insulative material.

15. The apparatus of claim 14, wherein the at least one thermally insulative material comprises air.

16. The apparatus of claim 13, wherein the second distance is at least 1 millimeter less than the first distance.

17. The apparatus of claim 16, wherein the portion comprises a central portion of the first housing and the first housing further comprises a peripheral portion encircling the central portion, the peripheral portion comprising the at least one energy transmission coil.

18. The apparatus of claim 13, further comprising at least one low loss driver electrically connected with the at least one energy transmission coil.

19. The apparatus of claim 18, further comprising a second housing separate from the first housing, the at least one low loss driver in the second housing.

20. The apparatus of claim 19, wherein the at least one energy transmission coil and/or the second housing comprises a controllably adjustable capacitor and/or inductor configured to tune a resonant frequency of the at least one energy transmission coil.

21. The apparatus of claim 13, further comprising a sensor configured to generate signals indicative of a position and/or an orientation of the first housing and circuitry configured to receive the signals and to adjust operation of the at least one energy transmission coil in response to the signals.

22. A method comprising:
placing a housing comprising a magnetic material and a power transmitting coil over a portion of a recipient's tissue overlying a power receiving coil beneath the tissue such that a region between the power transmitting coil and the recipient's tissue comprises at least one thermally insulative material, the magnetic material closer to the tissue than is the power transmitting coil; and
wirelessly transferring power from the power transmitting coil to the power receiving coil via a radio-frequency (RF) link.

23. The method of claim 22, wherein the power transmitting coil has a quality factor in a range of 30 to 250.

24. The method of claim 22, wherein the power transmitting coil and the power receiving coil have a magnetic coupling factor less than 0.3.

25. The method of claim 22, wherein a distance between the power transmitting coil and the power receiving coil is at least one millimeter greater than a thickness of the tissue between the magnetic material and the power receiving coil.

26. The method of claim 22, wherein a first distance between the magnetic material and an outer surface of the portion of the recipient's tissue overlying the power receiving coil is less than a second distance between the power transmitting coil and the outer surface of the portion of the recipient's tissue overlying the power receiving coil.
